(19) United States
(12) Patent Application Publication
(10) Pub. No.: US 2003/0170238 A1
(43) Pub. Date: Sep. 11, 2003

(54) Re-activated T-cells for adoptive immunotherapy

(76) Inventors: Micheal L. Gruenberg, Poway, CA (US); Surendra Chavan, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/246,647

(22) Filed: Sep. 17, 2002

Related U.S. Application Data
(63) Continuation-in-part of application No. 10/094,667, filed on Mar. 7, 2002.

(51) Int. Cl. A61K 39/395

(52) U.S. Cl. 424/144.1; 424/93.7

(57) ABSTRACT

A method for increasing the cytokine production of T-cells intended for use in adoptive immunotherapy is provided. The method improves adoptive immunotherapy methods where the efficacy of the treatment is dependent, at least in part, on the amount of cytokine production from the cells. In practicing the method, ex-vivo produced T-cells intended for use in adoptive immunotherapy treatment protocols are allowed to rest after harvest and then are re-activated just prior to infusion.

### RELATED APPLICATIONS

This application is a continuation-in-part of U.S. application Ser. No. 10/094,667, filed Mar. 7, 2002, to Micheal Gruenberg, entitled “RE-ACTIVATED T-CELLS FOR ADOPTIVE IMMUNOTHERAPY.” The subject matter of this application is incorporated by reference in its entirety.

This application is related to U.S. application Ser. No. 08/506,668, converted to U.S. provisional application Serial No. 60/044,693, now abandoned; pending U.S. application Ser. Nos. 08/700,565, 09/127,411, 09/127,142, 09/127,138, 09/127,141, 09/824,906, and International PCT application No. WO 97/05239. This application is also related to U.S. application Ser. No. 09/957,194, filed Sep. 19, 2001, to Micheal Gruenberg, entitled “The Adoptive Immunotherapy,” to U.S. provisional application Serial No. 60/322,626, filed September 17, 2001, entitled “Closed Sterile System Devices and Methods”, and to International PCT application No. PCT/US02/______ (attorney Docket No. 24731-508PC), filed the same day herewith. The subject matter of each of these applications is incorporated by reference in its entirety.

### FIELD OF THE INVENTION

Methods and compositions for adoptive immunotherapy are provided. In particular, methods for the re-activation of rested primed T-cells prior to infusion, such as for use in adoptive immunotherapy treatments are provided.

### BACKGROUND

The immune system is designed to eradicate a large number of pathogens, as well as tumors, with minimal immunopathology. When the immune system becomes defective, however, numerous disease states result. Immunotherapy is an emerging treatment modality that seeks to harness the power of the human immune system to treat disease. Immunotherapy seeks to either enhance the immune response in diseases characterized by immunosuppression or suppress the immune response in subjects with diseases characterized by an overactive immune response.

One immunotherapy method is a type of cell therapy called adoptive immunotherapy. A cell therapy is a drug whose active ingredient is wholly or in part a living cell. Adoptive immunotherapy is a cell therapy that involves the removal of immune cells from a subject, the ex-vivo processing (i.e., activation, purification and/or expansion of the cells) and the subsequent infusion of the resulting cells back into the same or different subject.

Examples of adoptive immunotherapy include methods for producing and using LAK cells (Rosenberg U.S. Pat. No. 4,690,915), TIL cells (Rosenberg U.S. Pat. No. 5,126,132), cytotoxic T-cells (Cai, et al U.S. Pat. No. 6,255,073; Celis, et al. U.S. Pat. No. 5,846,827), expanded tumor draining lymph node cells (Terman U.S. Pat. No. 6,251,385), various preparations of lymphocytes (Bell, et al U.S. Pat. No. 6,194,207; Ochoa, et al. U.S. Pat. No. 5,443,983; Riddell, et al. U.S. Pat. No. 6,040,180; Babbitt, et al. U.S. Pat. No. 5,766,920; Bolton U.S. Pat. No. 6,204,058), CD8+ TIL cells (Figlin et al. (1997) Journal of Urology 158:740), CD4⁺ T-cells activated with anti-CD3 monoclonal antibody in the presence of IL-2 (Nishimura (1992) J. Immunol. 148:285), T-cells co-activated with anti-CD3 and anti-CD28 in the presence of IL-2 (Garlie et al. (1999) Journal of Immunotherapy 22:336), antigen-specific CD8+ CTL T-cells produced ex-vivo and expanded with anti-CD3 and anti-CD28 monoclonal antibodies (mAb) in the presence of IL-2 (Oelke et al. (2000) Clinical Cancer Research 6:1997), and the first injection of irradiated autologous tumor cells admixed with Bacille Calmette-Guérin (BCG) to vaccinate subjects followed seven days later by recovery of draining lymph node T-cells which are activated with anti-CD3 mAb followed by expansion in IL-2 (Chang et al. (1997) Journal of Clinical Oncology 15:796).

Adoptive immunotherapy treatments have infrequent and sporadic efficacy. Protocols that require concomitant infusion of IL-2 or other cytokines are associated with high toxicity. The reasons for the infrequent and sporadic efficacy of these treatments are not clearly understood. There is a need to identify and solve these problems in order to increase the efficacy of adoptive immunotherapy protocols. Accordingly, it is an object herein to identify such problems and to solve them.

### SUMMARY

It is shown herein that a problem encountered in immunotherapy protocols is that by the time the cells intended for therapy are administered, they exhibit diminished cytokine production and viability. Provided herein is a solution to this problem as well as the problem of suppressed cytokine production from T-cells activated in a tumor microenvironment. Hence provided herein are methods for solving these problems, and also methods and compositions for adoptive immunotherapy. It is found that these methods not only solve the problems, but improve the efficacy of any T-cells for adoptive immunotherapy.

Also provided is a method that results in enhanced cytokine production from cells in a variety of environments, including immunosuppressive environments, such as tumor microenvironments. The method involves: (1) exposing a composition containing T-cells to one or more ex-vivo activation cycles; (2) removing the T-cells from the activation stimulus for at least 24 hours, generally at least 48-72 hours, generally 72-120 hours; and (3) re-activating the T-cells within 24 hours prior to infusion, generally within 4 hours prior to infusion, particularly within 1 hour prior to infusion. The T-cells include any compositions of cells that have been prepared, such as compositions of Th1 cells prepared as described herein and in co-pending U.S. application Serial Nos. 10/071,016 and 09/957,194, and in International PCT application No. PCT/US02/______ (attorney Docket No. 24731-504PC), filed the same day herewith.

Also provided are methods for enhancing the cytokine production of T-cells intended for use in adoptive immunotherapy. The methods involve the production of primed T-cells from patient source biological material, the resting of the primed T-cells and the subsequent re-activation of the primed T-cells just prior to infusion into a patient. Any method for activation and reactivation can be used, including, but not limited to, exposing the T-cells to antigens in the context of MHCI or MHCII molecules, superantigens, combinations of primary and co-stimulatory activation compounds, polyclonal activating compounds, mitogenic monoclonal antibodies, autologous or allogeneic antigen presenting cells alone or in combination with antigens, as well as allogeneic peripheral blood mononuclear cells and allogeneic lymphocytes.

In one embodiment, the T-cells are allowed to rest by removing them from an activation stimulus for at least 48-72 hours, typically at least about 72-120 hours, and then reactivating the cells prior to infusion by labeling the cells, for example, with mitogenic mAbs, such as soluble anti-CD3 and anti-CD28 mAbs and then mixing the labeled cells with autologous mononuclear cells that are optionally enhanced in monocytes and granulocytes.

The autologous mononuclear cells act by immobilizing the mitogenic mAbs on the cells, providing an activation stimulus. The mixture of cells is then suspended, for example, in infusion medium (e.g., isotonic solutions such as normal saline, 5% dextrose, Plasma-Lyte (Baxter) and Normasol (Abbott) or, as provided herein, mixed with autologous plasma, and infused into a patient within 24 hours, generally within 4 hours, generally within about 1 hour. If infusion medium is used, it is optionally supplemented with calcium chloride as needed for proper T-cell activation.

In one embodiment, the T-cells are labeled (i.e., contacted) with anti-CD3 and anti-CD28 mAbs and cryopreserved until ready for use. Just prior to infusion, the cells are thawed and mixed with fresh autologous leukapheresis product. This type of procedure can be conducted under FDA-mandated Good Manufacturing Practices using, for example the Cell Therapy system described in co-pending U.S. provisional application Serial No. 60/322,626, filed Sep. 17, 2001.

It was found that cells taken off an activation stimulus for about 48-120 hours, generally 72-120 hours, and reactivated just prior to infusion produce significantly more cytokines than the same cells produced while on or exposed to the activation stimulus.

It was further found herein that cells reactivated ex-vivo continue to produce cytokines in an environment that simulates the immunosuppressive microenvironment of a tumor lesion. This is important as the cytokine repertoire at the tumor site is a determinant for successful immune responses against tumors. Immunosuppressive cytokines, such as IL-10 and TGF-beta, that are produced by tumors target and paralyze primed cells and represent a major obstacle in cancer immunotherapy of tumor-bearing hosts. Use of cells reactivated as described herein overcome this obstacle.

Thus, methods that results in extended viability and sustained cytokine production of T-cells formulated for use in adoptive immunotherapy is provided. One method is a method formulation. The cells are formulated in autologous plasma and infused within about 48 hours. To formulate the cells, T-cells that have been primed and rested are mixed with autologous plasma after harvest from ex-vivo culture medium and prior to re-infusion into the subject. In another, the cells are reactivated, such as by contacting them with immobilized activating antibodies formulated in infusion medium. Generally the cells are formulated at a density of at least about 10⁶ cells per ml or 10⁷ cells per ml or 10⁸ cells per ml or 10⁹ cells per ml or higher.

Also provided are the resulting compositions of T-cells produced by the methods provided. The T-cells are formulated, such as suspended, in autologous plasma or other suitable medium. Generally the cells are at densities suitable for immunotherapy, Also provided are compositions of cells suspended in autologous plasma. The reactivated T-cells are suspended in the plasma, or other suitable medium, at densities of at least about 10⁶ cells per ml or 10⁷ cells per ml or 10⁸ cells per ml or 10⁹ cells per ml or 10¹⁰ cells per ml or higher.

Thus, compositions of formulated T-cells intended for use in adoptive immunotherapy that provide enhanced cytokine production and are capable of producing pro-inflammatory cytokines in a tumor microenvironment are provided. Among the compositions are: (1) ex-vivo activated, primed T-cells labeled (i.e., bound) with mitogenic monoclonal antibodies (mAbs) mixed with peripheral blood monocytes (PBMC); (2) ex-vivo activated, primed T-cells labeled with mitogenic mAbs mixed with a composition of autologous cells enriched in cells bearing Fc receptors; and (3) ex-vivo activated, primed T-cells labeled with mitogenic monoclonal antibodies (mAbs) mixed with allogeneic or autologous professional antigen presenting cells (APC), such as dendritic cells, B-cells or macrophages.

Cells are generally formulated in autologous plasma in order to avoid any adverse effects of infusion medium and are re-activated. Alternatively, for reactivation or reactivated cells are formulated in an infusion medium, such as a commercial medium i.e., Plasma-Lyte (Baxter), other medium, such as such as normal saline and 5% dextrose that has been supplemented with calcium chloride. Formulation can be performed at the patient bedside. In certain embodiments, the cells are re-activated within 4 hours of infusion; the precise time frame may depend upon the cell type and other conditions and can be empirically determined.

### DETAILED DESCRIPTION

A. Definitions

Unless defined otherwise, all technical and scientific terms used herein have the same meaning as is commonly understood by one of ordinary skill in the art to which these inventions belong. All patents, applications, published applications and other publications referred to throughout the disclosure herein are incorporated by reference in their entirety.

As used herein, cell therapy is a method of treatment involving the administration of live cells. Adoptive immunotherapy is a treatment process involving removal of cells from a subject, the processing of the cells in some manner ex-vivo and the infusion of the processed cells into the same or different subject as a therapy.

As used herein, source biological material is the population of cells that are collected from a subject for further processing into an adoptive immunotherapy. Source material generally is mononuclear cells collected, for example, by leukapheresis.

As used herein, a composition containing “purified cells” means that at least 50%, typically at least 70%, of the cells in the composition are of the identified type. For example, a composition containing purified CD4+ cells is a composition in which at least 50% of the cells in the compositions are CD4+.

As used herein, infusion medium is an isotonic solution suitable for intravenous infusion. Any such medium known to those of skill in the art can be used. Examples of infusion medium include, but are not limited to, normal saline (NS), 5% dextrose (D5W), Ringer's Lactate, Plasma-Lyte and Normosol and any other commercially available medium or medium knonw to one of skill in the art.

As used herein, a professional antigen presenting cells (APC) include dendritic cells, B-cells and macrophages.

As used herein, formulating for infusion is the process of removing or harvesting the cells to be used in adoptive immunotherapy from a culture environment, then subsequently washing, concentrating and re-suspending the cells in infusion medium or in plasma as provided herein.

As used herein, peripheral blood monocytes (PBMC) include autologous and allogeneic cells.

As used herein, culture medium is any medium suitable for supporting the viability, growth, and/or differentiation of mammalian cells ex-vivo. Any such medium known to those of skill in the art. Examples of culture medium include, but are not limited to, X-Vivo15 (BioWhittaker), RPMI 1640, DMEM, Ham's F12, McCoys 5A and Medium 199. The medium can be supplemented with additional ingredients including serum, serum proteins, growth suppressing, and growth promoting substances, such as mitogenic monoclonal antibodies and selective agents for selecting genetically engineered or modified cells.

As used herein, an immunosuppressive tumor environment is the microenvironment created by cytokine production from tumor cells and infiltrating mononuclear cells. The sum total of cytokines create an environment that is capable of suppressing the effector functions of immune cells. Examples of immunosuppressive cytokines in a tumor microenvironment include IL-10 and TGF-beta.

As used herein, a resting T-cell means a T-cell that is not dividing or producing cytokines. Resting T-cells are small (approximately 6-8 microns) in size compared to activated T-cells (approximately 12-15 microns).

As used herein, a primed T-cell is a resting T-cell that has been previously activated at least once and has been removed from the activation stimulus for at least 48 hours. Primed T-cells usually have a memory phenotype.

As used herein, an activated T-cell is a T-cell that has received at least two mitogenic signals. As a result of activation, a T-cell will flux calcium which results in a cascade of events leading to division and cytokine production. Activated T-cells can be identified phenotypically, for example, by virtue of their expression of CD25. Cells that express the IL-2 receptor (CD25) are referred to herein as “activated”. A pure or highly pure population of activated cells typically express greater than 85% positive for CD25.

As used herein, source material is the population of cells that are collected from a subject for further processing into an adoptive immunotherapy. Source material generally is mononuclear cells collected, for example, by leukapheresis.

As used herein, a cell therapeutic refers to the compositions of cells that are formulated as a drug whose active ingredient is wholly or in part a living cell.

As used herein, immune cells are the subset of blood cells known as white blood cells, which include mononuclear cells such as lymphocytes, monocytes, macrophages and granulocytes.

As used herein, T-cells are lymphocytes that express the CD3 antigen.

As used herein, helper cells are CD4+ lymphocytes.

As used herein, regulatory cells are a subset of T-cells, most commonly CD4⁺ T-cells, that are capable of enhancing or suppressing an immune response. Regulatory immune cells regulate an immune response primarily by virtue of their cytokine secretion profile. Some regulatory immune cells can also act to enhance or suppress an immune response by virtue of antigens expressed on their cell surface and mediate their effects through cell-to-cell contact. Th1 and Th2 cells are examples of regulatory cells.

As used herein, effector cells are immune cells that primarily act to eliminate tumors or pathogens through direct interaction, such as, but not limited, through phagocytosis, perforin and/or granulozyme secretion and induction of apoptosis. Effector cells generally require the support of regulatory cells to function and also act as the mediators of delayed type hypersensitivity reactions and cytotoxic functions. Examples of effector cells are B lymphocytes, macrophages, cytotoxic lymphocytes, LAK cells, NK cells and neutrophils.

As used herein, T-cells that produce IFN-gamma, and not IL-4 upon stimulation are referred to as Th1 cells. Cells that produce IL-4, and not IFN-gamma, are referred to as Th2 cells. A method for identifying Th1 cells in a population of cells is to stain the cells internally for IFN-gamma. Th2 cells are commonly identified by internal staining for IL-4. In normal (i.e., subjects not exhibiting overt disease) individuals, generally only about 12-16% of the CD4+ cells stain positive for internal IFN-gamma after activation; less than 1% stain positive for IFN-gamma prior to activation. It is rare for a T-cell population to stain greater than 35% IFN-gamma positive. The cells resulting from a method described herein (and provided in co-pending U.S. application Ser. No. 09/957,194, filed Sep. 19, 2001), stain greater than 70% positive and often greater than 90% positive for IFN-gamma.

As used herein, a pure or highly pure population of Th1 cells is a population that stains greater than 70% positive for internal IFN-gamma and does not produce greater than about 26 pg/ml/10⁶ cells of IL-4 in a 24 hour period. In most instances, they do not produce greater than about 6 pg/ml/10⁶ cells of IL-4 in a 24 hour period.

As used herein, a memory cell is a T-cell that expresses CD45RO and not CD45RA. A pure or highly pure population of memory cells expresses greater than 70%, generally greater than 80%, and even greater than 90% or 95% positive for CD45RO.

As used herein, a cell that has the ability to traffic to a tumor or other site of inflammation upon infusion, is a T-cell with an activated (CD25+) memory (CD45RO+) phenotype that expresses adhesion molecules, such as CD44 and does not expresses CD62L. A pure or highly pure population of memory cells with the ability to traffic to a tumor or other site of inflammation upon infusion is greater than 70%, generally greater than 90% or 95% positive for CD44, and less than about 25%, including less than 5%, positive for CD62L.

As used herein, T-cells intended for adoptive immunotherapy refer to any T-cells that have been treated for use in adoptive immunotherapy. Examples of such cells include any T-cells prepared for adoptive immunotherapy and, include but are not limited to, for example Th1 cells (co-pending U.S. application Ser. No. 09/957,194), co-stimulated T-cells (Lums, et al. (2001) J Immunother 25:408), polyclonal and antigen-specific CTL (Maus et al. (2002) Nat. Biotechnol. 20:143), co-stimulated CD4+cells (Levine et al. (2002) Nat. Med 8:47), CML-specific T-cells (Muller et al. (2002) J Immunother. 24:482), soluble tumor antigen induced CTL (Li et al. (2001) Zhonghua Wai Ke Za Zhi 39:619), anti-cervical cancer CTL (Chiriva-Internati et al. (2002) Eur. J. Immunol. 32:30), tumor associated lymphocytes (Schuler et al. (2001) J. Exp. Med. 194:1767), EBV-specific T-cells (Savoldo et al. (2002) J. Immunol. 168:909; Hague et al. (2001) Transplantation 72:1399), CML-specific T-cells (Muller et al. (2001) J. Immunother. 24:482), CTL against lung cancer (Hiraki et al. (2001) Anticancer Res.21:2561; So et al. (2001) Jap J. Clin. Oncol.31:311), anti-leukemia CTL (Montagna et al. (2001) Blood 98:3359), ex-vivo activated lymph node cells (Plautz et al. (2001) Cancer Chemother Biol Response Modif 19:327), interferon-gamma enhanced T-cells (Becker et al. (2001) Nat Med. 7:1159), pharmacologically-activated lymph node cells (Bear et al. (2001) Cancer Immunol Immunother. 50:269), gamma-delta T-cells (Chen et al. (2001) Int. Arch. Allergy Immunol. 125:256), CMV-specific CTL (Szmania et al. (2001) Blood 98:505; Cho et al. (2001) J. Immunother. 24:242), activated T-cells (Chin et al. (2001) J Surg Res 98:108), pre-immunized effector cells (Morecki et al. (20010 J. Immunother 24:114), cytotoxic T-cells (U.S. Pat. No. 6,255,073; U.S. Pat. No. 5,846,827), expanded tumor draining lymph node cells (U.S. Pat. No. 6,251,385), various preparations of lymphocytes (U.S. Pat. No. 6,194,207; U.S. Pat. No. 5,443,983; U.S. Pat. No. 6,040,180; U.S. Pat. No. 5,766,920; U.S. Pat. No. 6,204,058), CD8+ TIL cells (Figlin et al. (1997) Journal of Urology 158:740), CD4⁺ T-cells activated with anti-CD3 monoclonal antibody in the presence of IL-2 (Nishimura (1992) J. Immunol. 148:285), T-cells co-activated with anti-CD3 and anti-CD28 in the presence of IL-2 (Garlie et al. (1999) Journal of Immunotherapy 22:336), antigen-specific CD8+ CTL T-cells produced ex-vivo and expanded with anti-CD3 and anti-CD28 monoclonal antibodies (mAb) in the presence of IL-2 (Oelke et al. (2000) Clinical Cancer Research 6:1997), and the first injection of irradiated autologous tumor cells admixed with Bacille Calmette-Guérin (BCG) to vaccinate subjects followed seven days later by recovery of draining lymph node T-cells which are activated with anti-CD3 mAb followed by expansion in IL-2 (Chang et al. (1997) Journal of Clinical Oncology 15:796).

As used herein, activating proteins are molecules that when contacted with a T-cell population cause the cells to proliferate. Reference to activating proteins thus encompasses the combination of proteins that provide the requisite signals, which include an initial priming signal and a second co-stimulatory signal. The first signal requires a single agent, such as anti-CD3 monoclonal antibody (mAb), anti-CD2 mAb, anti-TCR mAb, PHA, PMA, and other such signals. The second signal requires one or more agents, such as anti-CD28 mAb, anti-CD40L, anti-CD99, anti-CD4 mAb, cytokines, feeder cells and other such signals. Thus activating proteins include combinations of molecules including, but are not limited to: cell surface protein specific mAbs, fusion proteins containing ligands for a cell surface protein, or any molecule that specifically interacts with a cell surface receptor on a T-cell and directly or indirectly causes that cell to proliferate.

As used herein, a mitogenic mAb is an activating protein that is a monoclonal antibody specific for a T-cell surface expressed protein that when contacted with a cell directly or indirectly provides one of the at least two requisite signals for T-cell mitogenesis. Suitable mitogenic mAbs induce T-cell doubling times of 24 h to 48 h.

As used herein, a cytokine is a factor produced from a cell that has biological activity. A lymphokine is a cytokine produced by lymphocytes. Interleukins and interferons are examples of lymphokines.

As used herein, exogenous cytokines, refer to cytokines that are added to a sample or cell preparation. They do not include cytokines produced by the cells in a sample or cell preparation in vitro, in vivo or ex vivo. Hence preparing cells in the absence of exogenous cytokines, refers to preparation without adding additional cytokines to those produced by the cells.

As used herein, a composition containing a clinically relevant number or population of immune cells is a composition that contains at least 10⁹, typically greater than 10⁹, at least 10¹⁰ cells, and generally more than 10¹⁰ cells. The number of cells will depend upon the ultimate use for which the composition is intended as will the type of cell. For example, if Th1 cells that are specific for a particular antigen are desired, then the population will contain greater than 70%, generally greater than 80%, 85% and 90-95% of such cells. For uses provided herein, the cells are generally in a volume of a liter or less, can be 500 mls or less, even 250 mls or 100 mls or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells.

As used herein, a clinically relevant number of activated polyclonal Th1 memory cells is a composition containing a clinically relevant number or population of immune cells where a substantial portion, greater than at least about 70%, typically more than 80%, 90%, and 95%, of the immune cells are activated polyclonal Th1 memory cells.

As used herein, polyclonal means cells derived from two or more cells of different ancestry or genetic constitution. A polyclonal T-cell population is a population of T-cells that express a mixture of T cell receptor genes with no one T cell receptor gene dominating the population of cells.

As used herein, predominant means greater than about 50%.

As used herein, highly pure means greater than about 70%, generally greater than 75% and can be as pure as 85%, 90% or 95% or higher in purity. A highly pure population of Th1 cells, as used herein, is typically a population of greater than 95% CD3+, CD4⁺ T-cells that stain greater than about 70% positive for internal IFN-gamma and do not produce detectable amounts of IL-4 when assayed by ELISA (i.e., less than 26 pg/ml/10⁶ cells). Internal staining for IL-4 is generally below 10% and most often below 5%. Occasionally higher numbers are observed. This is often an artifact of the detection technique, as cells that die by apoptosis will stain positive for internal IL-4. Measurement of secretion into supernatants controls for this artifact. The amount of IFN-gamma detected by ELISA is generally in excess of 1 ng/ml/10⁶ cells and in the range of 1 ng/ml to 26 ng/ml per 10⁶ cells, but can be greater than 26 ng/ml per 10⁶ cells.

As used herein, a combination refers to two component items, such as compositions or mixtures, that are intended for use either together or sequentially. The combination may be provided as a mixture of the components or as separate components packaged or provided together, such as in a kit.

As used herein, colloidal size beads are particles of a size that form a colloid upon mixing with a liquid, such as an aqueous composition. Such particles typically have an a size where the largest dimension is about 0.01 to 2 microns. For purposes herein, it refers to the size of the particles produced in the method of Example 1G.

As used herein, effector cells are mononuclear cells that have the ability to directly eliminate pathogens or tumor cells. Such cells include, but are not limited to, LAK cells, MAK cells and other mononuclear phagocytes, TILs, CTLs and antibody-producing B cells and other such cells.

As used herein, immune balance refers to the normal ratios, and absolute numbers, of various immune cells and their cytokines that are associated with a disease free state. Restoration of immune balance refers to restoration to a condition in which treatment of the disease or disorder is effected whereby the ratios of regulatory immune cell types or their cytokines and numbers or amounts thereof are within normal range or close enough thereto so that symptoms of the treated disease or disorder are ameliorated. The amount of cells to administer can be determined empirically, or, such as by administering aliquots of cells to a subject until the symptoms of the disease or disorder are reduced or eliminated. Generally a first dosage will be at least 10⁹-10¹⁰ cells. In addition, the dosage will vary depending upon treatment sought. As intended herein, about 10⁹ is from about 5×10⁸ up to about 5×10⁹; similarly about 10¹⁰ is from about 5×10⁹ up to about 5×10¹⁰, and so on for each order of magnitude. Dosages refer to the amounts administered in one or in several infusions.

As used herein, therapeutically effective refers to an amount of cells that is sufficient to ameliorate, or in some manner reduce the symptoms associated with a disease. When used with reference to a method, the method is sufficiently effective to ameliorate, or in some manner reduce the symptoms associated with a disease.

As used herein, a subject is a mammal, typically a human, including patients.

As used herein, mononuclear or lymphoid cells (the terms are used interchangeably) include lymphocytes, macrophages, and monocytes that are derived from any tissue or body fluid in which such cells are present. In general lymphoid cells are removed from an individual who is to be treated. The lymphoid cells may be derived from a tumor, peripheral blood, or other tissues, such as the lymph nodes and spleen that contain or produce lymphoid cells.

As used herein, a therapeutically effective number is a clinically relevant number of immune cells that is at least sufficient to achieve a desired therapeutic effect, when such cells are used in a particular method. Typically such number is at least 10⁹, and generally 10¹⁰ or more. The precise number will depend upon the cell type and also the intended target or result and can be determined empirically.

As used herein, a disease characterized by a lack of Th1 cytokine activity refers to a state, disease or condition where the algebraic sum of cytokines in a specific microenvironment in the body or in a lesion(s) or systemically is less than the amount of Th1 cytokines present normally found in such microenvironment or systemically (i.e., in the subject or another such subject prior to onset of such state, disease or condition). The cytokines to assess include IFN-gamma, IL-2, and TNF-beta. The precise amounts and cytokines to assess depend upon the particular state, disease or condition. Thus, the diseases for which the cells have therapeutic application include, but are not limited to, cancer, infectious diseases, allergic diseases and diseases characterized by overactive humoral immunity (such as in systemic lupus erythematosus).

As used herein, diseases characterized by a Th2-dominated immune response are characterized by either a suppressed cellular immune response or excessive humoral response.

As used herein, a disease characterized by an excess of Th2 cytokine activity refers to a state, disease or condition where the algebraic sum of cytokines in a specific microenvironment in the body or in a lesion(s) or systemically is predominantly of the Th2 type, dominated by IL-4 and/or IL-10 and/or TGF-beta. Diseases, states or conditions that exhibit enhanced Th2 responses include infectious diseases such as, but are not limited to, chronic hepatitis C virus infection, leprosy toxoplasmosis infection and AIDS. Imbalance in favor of Th2 cells also occurs in asthma and lupus and other diseases that exhibit suppressed cellular immunity.

As used herein, treatment means any manner in which the symptoms of a condition, disorder or disease are ameliorated or otherwise beneficially altered. Treatment also encompasses any pharmaceutical use of the compositions herein.

As used herein, a vaccine is a composition that provides protection against a viral infection, cancer or other disorder or treatment for a viral infection, cancer or other disorder. Protection against a viral infection, cancer or other disorder will either completely prevent infection or the tumor or other disorder or will reduce the severity or duration of infection, tumor or other disorder if subsequently infected or afflicted with the disorder. Treatment will cause an amelioration in one or more symptoms or a decrease in severity or duration. For purposes herein, a vaccine results from co-infusion (either sequentially or simultaneously) of an antigen and a composition of cells produced by the methods herein.

As used herein, amelioration of the symptoms of a particular disorder by administration of a particular composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as flow cytometry, used by those of skill in the art to assess such purity, or sufficiently pure such that further purification would not detectably alter the physical and chemical properties, such as biological activities, of the substance. Methods for purification of the immune cells to produce substantially pure populations are known to those of skill in the art. A substantially pure cell population, may, however, be a mixture of subtypes; purity refers to the activity profile of the population. In such instances, further purification might increase the specific activity of the cell population.

As used herein, biological activity refers to the in vivo activities of immune cells or physiological responses that result upon in vivo administration of a cell, composition or other mixture. Biological activity, thus, encompasses therapeutic effects and pharmaceutical activity of such cells, compositions and mixtures.

Although any similar or equivalent methods and materials can be employed in the practice of the methods and cells provided herein, exemplary embodiments are described.

B. Problems With Prior Methods and Solutions Provided Herein

The efficacy of adoptively transferred T-cells is dependent, in part, on their ability to produce cytokines and chemokines, either systemically or in or near the disease location. Cytokines and chemokines are chemical messengers that act to regulate the development and expression of the broad array of immune responses that are mounted against a variety of pathogens and tumors. The types, amounts and ratios of cytokines and chemokines produced at a site of inflammation are determinants of the types of cells which regulate and participate in innate and adaptive immune responses. The cytokines produced also can act by directly mediating anti-tumor or antimicrobial effector activities. Numerous immune cells are responsive to cytokines, including dendritic cells, macrophages and other antigen-presenting cells, T cells and B cells. Accordingly, in order to optimize the effectiveness of adoptive immunotherapy protocols, it is essential to optimize the amounts and types of cytokines produced by the cells used as therapy.

Thus, it is shown herein that prior methods for formulating T-cells for use in adoptive immunotherapy result in compositions of cells that have significantly diminished viability and cytokine production. Since the viability of the infused cells and the ability to produce cytokines is essential for therapeutic efficacy of these cells, this may be one reason to explain the low efficacy of prior methods and compositions.

Prior methods for formulating T-cells for use in adoptive immunotherapy generally harvest cells from an ex-vivo culture environment, subsequently wash and resuspend the harvested cells in an isotonic infusion medium. As described herein, however, storage of T-cells formulated for infusion in excess of 24 hours results in significant loss of viability. This can be solved by formulating the cells in autologous plasma. Cells formulated in autologous plasma can be stored for 48 h prior to infusion without loss of viability.

As described herein, prior methods for formulating T-cells for use in adoptive immunotherapy result in compositions of cells that are primed for cytokine production, but do not produce significant amounts of cytokines. These primed cells require in vivo activation in order to produce cytokines. It is also shown herein that primed T-cells do not produce pro-inflammatory cytokines when activated in an environment that simulates the immunosuppressive microenvironment found in tumors. This may be another reason to explain the low efficacy of prior methods and compositions.

Thus, problems that limit the efficacy of adoptive immunotherapy protocols are identified herein. These problems include: (1) cells suspended in infusion medium rapidly lose viability and the ability to produce cytokines; (2) cytokine production from cells processed for adoptive immunotherapy wanes in time; and (3) cells processed for adoptive immunotherapy do not produce cytokines in an environment that simulates the immunosuppressive environment of a tumor.

It shown herein that prior adoptive immunotherapy protocols result in the infusion of cells that do not constitutively produce cytokines.

Adoptive immunotherapy protocols involve removing source biological material from a patient, processing the cells ex-vivo and then formulating the cells for infusion. Generally, the cells are formulated by first harvesting them from a culture medium which was formulated for growth and maintenance of the cells, and then washing and concentrating the cells in a medium and container system suitable for infusion. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium is often supplemented with human serum albumen.

The FDA considers the cells in adoptive immunotherapy products to be a “drug” as that term is defined under 21 United States Code (USC) 321(g). As a drug, adoptive immunotherapy products must be manufactured under Good Manufacturing Practices (GMP). Part of these GMP requirements are the testing of each lot of cells used in the adoptive immunotherapy protocols, including analysis of cell yield, viability, function (including cytokine production), purity and sterility.

It was determined herein that prior protocols for testing lots of cells are flawed. The samples of cells taken for GMP testing and analysis are generally removed prior to the formulation step, thus the cells are sampled from cells incubated in culture medium and not infusion medium. Since it is the cells that are in the infusion medium which are delivered to the patient and not the cells in culture medium, the testing of cells from culture medium will fail to detect any changes in the cells which may have occurred while being suspended in infusion medium.

It was found herein that significant changes occur in cells suspended in infusion medium compared to cells suspended in culture medium. The formulated cells are generally kept in the infusion medium for several hours prior to infusion, typically for 24-48 hours. Changes in both viability and cytokine production occur over this period of time.

It is found herein that cells in infusion medium lose viability rapidly. The infusion medium does not contain supportive nutrients to maintain the cells. It is found that cells suspended at densities of 10 to 100 million per ml of infusion medium lose 20-60% of their viability in 24 hours at room temperature. The loss of viability was a function of cell density, time in suspension and temperature. The higher the cell density, the more rapid the loss in viability. The higher the temperature, the more rapid the loss of viability. These parameters are rarely, if ever, controlled in prior adoptive immunotherapy protocols.

The determination of the types and amounts of cytokine production from cells is a standard test for predicting the function of cells used in adoptive immunotherapy treatments. The production of cytokines is also generally determined from cell samples derived from culture medium and not infusion medium. Further, the sampled cells, when tested for cytokine production capability, are generally activated ex-vivo in culture medium for this determination because harvested cells do not constitutively produce cytokines (see for example, Lum et al. (2001) J Immunother 24:408,413). This testing method does not reflect the status of the cells actually infused.

It was also found that even cells prepared according to the methods described in described in co-pending U.S. application Ser. No. 09/957,194 that constitutively produced cytokines when harvested from culture medium ceased to produce cytokines after suspension in typical infusion medium within 24 hours. Since cells processed for use in adoptive immunotherapy are usually infused into a patient 24-48 h after suspension in infusion medium, the loss of viability and cytokine production may be a reason for the inconsistent results and limited efficacy of these methods.

Thus, in order to maximize the effectiveness of adoptive immunotherapy protocols it is desirable to enhance the cytokine production of the cells for infusion. Cytokines regulate the initiation, maintenance and suppression of immune responses against foreign antigens and tumors. This regulation is mediated by CD4+ helper cells that are subdivided into distinct subsets based upon the type of cytokines they produce. Th1 cells produce IFN-gamma and promote cell-mediated immune responses and viral neutralizing antibody responses of the IgG2a isotype, while Th2 cells produce IL-4 and stimulate B-cell proliferation and differentiation promoting predominantly IgG1 and IgE antibody production.

There are a variety of methods for producing cells for adoptive immunotherapy. For example, co-pending U.S. application Serial Nos. U.S. application Ser. Nos. 08/700,565, 09/127,411, 09/127,142, 09/127,138, 09/127,141, 09/824,906, and International PCT application No. WO 97/05239 provide methods for producing compositions containing clinically relevant numbers of T-cells, and co-pending U.S. application Ser. No. 09/957,194, provides methods for preparation of highly pure compositions of Th1 cells and the resulting compositions. Briefly, U.S. application Ser. No. 09/957,194 provides a method for producing substantially pure compositions of Th1 cells by collecting source material from a subject; purifying T-cells from the source material; and stimulating or activating the T-cells a minimum of 3 times at 2-4 day intervals, such as by contacting the cell with immobilized anti-CD3 and anti-CD28 mAb. The frequency of the restimulation must be every 2-3 days and the restimulation must be repeated at least 3 and typically 4 times in order to obtain a pure population of activated Th1 memory cells. The resulting cells constitute a highly pure population of polyclonal Th1 memory cells. The cells are processed in the absence of any exogenous cytokines.

Cells intended for immunotherapy are packaged, for example, in saline supplemented with human serum albumin and then shipped to the patient. Generally there is at least a 24 hour delay from removal from culture and infusion. It is found herein that total viability is only about 40% to 50%, and there is no cytokine production. Low viability and low cytokine production is likely a heretofore unrecognized problem experienced in all adoptive immunotherapy protocols. Generally the cells are assayed before introducing them into saline, but once the cells formulated in infusion medium and packaged in an IV bag, they are not assayed. It has not been recognized that the cells have low viability and are not producing cytokines.

It was found herein that if T-cells are prepared and are then rested by removing them from an activation stimulus for another 24-120 hours, particularly 48-120 hours, generally about 72 hours, and then reactivated, the cells will ultimately produce about 10-fold more cytokines than before they rested. Hence in embodiments of the methods herein, cells are harvested, rested for 24-120 hours, typically 48-120 hours, generally 72 to 96 hours, and then frozen. Prior to infusion into a patient the cells are formulated in autologous plasma and are reactivated.

C. Activation

In accord with the methods herein, T-cells are treated to reactivate them just prior to re-infusion, typically within four hours. It is contemplated herein that any method for activation of T-cells can be used just prior to infusion. Such activation should be performed no more than about 24 hrs, and is typically 8, 6, or 4 hours before infusion. The best time for infusion, should be after the cells are re-activated but before cytokine production increases substantially, since infusion of cells that are producing large amounts of cytokines may be toxic. This timing can be determined empirically by activating the cells and measuring cytokine production as a function of time. For the exemplified cells this time period is about 4 hours after activation (see, e.g., EXAMPLES, for an exemplary time course).

1) General Methods for Activating T-Cells

In order for T-cells to proliferate, they require two separate signals. The first signal is generally delivered through the CD3/TCR antigen complex on the surface of the cells, and the second is generally provided through the IL-2 receptor. For cells used in adoptive immunotherapy, IL-2 is generally used as the second signal. In order to bypass the IL-2 signal, combinations of mAb can be used for activation. The mAb can be in the soluble phase or immobilized on plastic or other solid surfaces such as on magnetic beads.

(a) First Signal

To provide the first signal, cells are generally activated with mAb to the CD3/TCR complex, but other suitable signals, such as, but not limited to, antigens, super antigens, polyclonal activators, anti-CD2 and anti-TCR antibodies, can be used. Other suitable agents can be empirically identified. Immobilized or cross-linked anti-CD3 mAb, such as OKT3 or 64.1, can activate T-cells in a polyclonal manner (see, Tax, et al. (1983) Nature 304:445). Other polyclonal activators, however, such as phorbol myristate acetate also can be used (see, e.g., Hansen, et al. (1980) Immunogenetics 10:247).

Monovalent anti-CD3 mAb in the soluble phase also can be used to activate T-cells (see, Tamura et al. (1992) J. Immunol. 148:2370). Stimulation of CD4+cells with monovalent anti-CD3 mAb in the soluble form is preferable for expansion of Th2 cells, but not Th1 cells (see, deJong, et al. (1992) J. Immunol. 149:2795). Soluble heteroconjugates of anti-CD3 and anti-T-cell surface antigen mAb can preferentially activate a particular T-cell subset (see, e.g., Ledbetter, et al. (1988) Eur. S. Immunol. 18:525). Anti-CD2 mAb can also activate T-cells (see, Huet, et al. (1986) J. Immunol. 137:1420). Anti-MHC class 11 mAb can have a synergistic effect with anti-CD3 in inducing T-cell proliferation (see, Spertini et al. (1992) J. Immunol. 149:65). Anti-CD44 mAb can activate T-cells in a fashion similar to anti-CD3 mAb. See, Galandrini, et al. (1993) J. Immunol. 150:4225)

(b) Second Signal

A variety of mAb singly or in combination can provide the second signal for T-cell activation. Immobilized mAb or fusion proteins which interact with co-stimulatory molecules such as CD28, CD134 (OX40) and CD137 (4-1 BB) or adhesion molecules on T-cells such as CD54 (ICAM-1), CD11a/CD18 (LFA-1) and CD49d/CD29 (VLA-4) singly or in combination can provide second signals for activation.

To determine the combination of mAbs or proteins that optimally induce sustained regulatory cell proliferation, a screening procedure using combinations of these mAbs or proteins is used. The cells are incubated with various combinations of these substances and screened for growth by analysis of ³H-thymidine incorporation or equivalent methods. The group demonstrating the best growth characteristics is selected for use.

2) Exemplary Methods for Re-Activating the T-Cells Prior to Infusion

Any method for activating T-cells can be employed. In most instances, since the cells are to be reactivated at the patient bedside or on site. The method is conducted in a manner the maintains sterile conditions, such as those required by Good Manufacturing Practices (GMP). Methods for reactivation are provided herein.

a) In one method, a patient is leukapheresed, and mononuclear cells, which are enriched in granulocytes and monocytes, are collected. At same time, the frozen cells are labeled with anti-CD3/CD28 antibodies, preferably IgG1, mixed with the enriched mononuclear cells. The granulocytes and monocytes have Fc receptors that bind with high avidity to Fc portion of IGg1. Therefore they deliver a signal to the cells, activating them. The resulting cytokine profile from the cells is another log higher than when they are activated with bead-bound monoclonal antibodies. In addition, the cells activate the monocytes and granulocytes to produce cytokines, such as IL-12, which are macrophage, not T-cell, products.

The resulting mixture of cells produce so much cytokine that they could be cytotoxic. It was found, however that there are no measurable cytokines within the first 4 hours of activation, and that the peak of cytokine production is at 24 hrs. Therefore, the cell composition is infused within four hours after activation. If, for example, the cells are memory cells (see, e.g., co-pending U.S. application Ser. No. 09/957,194), they traffic to tumors and sites of inflammation, and start producing cytokines at the targeted site(s).

b) Another method for activating T-cells for use in adoptive immunotherapy protocols is to incubate the cells with immunomagnetic beads conjugated with anti-CD3/anti-CD28 mAbs. Cells activated in this manner must be removed from the beads prior to infusion, as the beads are not intended for human infusion. Typically, the conjugated beads are separated from the cells using a magnet. The initial interaction between the conjugated beads and the cells in strong. Attempts to remove the conjugated beads from the cells within 24 hours, results in significant cell death, presumably due to damage to the cell membranes as the beads are pulled off the cells. After 24 hours, and preferably after 48 hours, the interaction between the conjugated beads and the cells weaken and the cells can be readily separated without significant loss of viability. However, cells that are removed from the conjugated beads after 24-48 hours produce diminished amounts of cytokines.

c) In accord with the methods provided herein, activted T-cells are removed from the conjugated beads after 48 hours and incubated without activating stimulus for an additional 24-48 hours. When these resting cells are reactivated, they produce at least about 2-10-fold, generally at least about 5-20-fold, more cytokine than cells that were not rested and reactivated. In addition, rested and reactivated cells continue to produce cytokines for at least 96 hours after restimulation. Non-rested, stimulated cells only produce cytokines for 48 hours.

Thus, as provided herein, to advantageously employ cells for adoptive immunotherapy protocols, cells are reactivated just prior to infusion into a patient. Reactivation can be effected by any method of activation. Mitogenic mAbs, however, require immobilization in order to deliver an activation signal to T-cells, which is provided by beads with immobilized antibodies. Conjugated beads cannot be used for activation prior to reinfusion, since they readily can not be removed when added just prior to infusion and conjugated beads can not be infused in high quantity to a patient. Accordingly, an alternative activation method is required.

Immobilization of mitogenic mAbs for use in the methods herein can be accomplished by labeling T-cells intended for infusion with anti-CD3/anti-CD28 mAb, such as antibodies of the IgG1 subclass, and subsequently mixing the labeled cells with autologous mononuclear cells, generally enriched in granulocytes and macrophages. Fc gammã-RI receptors expressed on neutrophils, monocyte/macrophages and eosinophils have a high avidity for the Fc portion of antibodies, especially of the IgG1 or IgG3 subclasses.

The mixed cells can be suspended in infusion medium and immediately infused into a patient. One way to do this is to mix the labeled cells with autologous mononuclear cells during a leukapheresis procedure. In this manner, the cells are not required to be suspended in infusion medium prior to infusion.

Alternatively, the cells can be mixed with anti-CD3/anti-CD28-conjugated colloidal size particles, dextran coated paramagnetic microbeads beads (Miltenyi Biotec, Auburn Calif.; see, U.S. Pat. No. 6,417,011; see EXAMPLES, below). Such micro-particles remain in suspension since they are colloidal in size. In addition, following binding to CD4 T cells are internalized or shed, as a result the activation signal through CD3 and CD28, is transient and not continuous, and the need to debead the product prior to infusion in patients is eliminated.

The following examples are included for illustrative purposes only and are not intended to limit the scope of the invention.

### EXAMPLE 1

Materials and Methods

A. Isolation of Human Lymphocytes.

Samples of buffy coats or leukapheresis products from normal donors and EDTA-preserved blood samples from advanced cancer subjects with a variety of indications and prior treatments were used. Human peripheral blood lymphocytes (PBMC) were isolated using a density gradient centrifugation procedure.

B. Characterization of PBMC Samples

Purified PBMC samples were characterized by immuno-phenotyping using flow cytometry. Briefly, cells were incubated with fluorochrome-labeled antibodies in the dark for 30 min., washed of excess antibodies and analyzed on FACSCalibur flow cytometer (BD Biosciences). Results of the analysis were expressed as percentages of total lymphocytes, monocytes, granulocytes, and also subsets of lymphocytes: B-cells, cytotoxic T lymphocytes, CD4 positive T-helpers, and NK cells. The subset of CD4 positive T cells was analyzed for the ratio between naive CD45RA positive cells and CD45RA negative memory cells.

C. Cytokine Profiling

To determine the ability of freshly purified CD4 positive cells to express IFN-gamma and IL-4 an intra-cellular cytokine (ICC) staining procedure using an Internal Cellular Cytokine (ICC) kit (BioErgonomics, St. Paul, Minn.) was performed. According to the manufacturer's recommendation, PBMC were stimulated for 20 h in T-cell activation medium, stained first by surface anti-CD4 antibodies, fixed, permeated and then stained with intracellular anti-IFN-gamma and anti-IL-4 antibodies. Samples were analyzed by flow cytometry and results were presented as percentages of IFN-gamma and IL-4 expressing cells in CD4 positive T cells subset.

D. Isolation of T-Cell Subpopulations

Isolation of specific T-cell subpopulations was performed using two different techniques: sort by flow cytometry on FACSCalibur and sort by combination of positive and negative immunomagnetic selection on AutoMacs (Miltenyi, Germany). To obtain cell samples with high purity, sort by flow cytometry was done. Briefly 4×10⁷ of PBMC were stained with anti-CD4 antibodies alone or in combination with anti-CD45RO antibodies, labeled with the corresponding fluorochrome. Subsets of CD4-positive, CD4-positive/CD45RO-negative and CD4-positive/CD45RO-positive cells were collected by sorting and used for expansion experiments. To obtain better yields with 5-10% lower purities, separation for further applications used immunomagnetic selection.

According to the manufacturer's recommendation, up to 2×10⁸ cells were incubated with anti-CD4 antibodies conjugated directly to magnetic microbeads and separated on magnetic columns. If needed, the second round of selection was performed using mouse anti-CD45RO antibodies in complex with goat anti-mouse antibodies conjugated to microbeads.

E. Activation of Cells

Sorted cells were plated into cell culture plates at starting concentrations of 1×10⁵ to 3×10⁵ cells/ml using ex vivo serum free cell culture medium (X-VIVO-15 from BioWhittaker) without supplementation. The cells were cultured for 12 days and were repeatedly activated using a combination of CD3/CD28 antibodies conjugated to magnetic beads (T-cell Expander, Dynal) every 3 days, starting from the day of sort (pursuant to the methods of co-pending U.S. application Ser. Nos. 10/071,016 and 09/957,194, and in International PCT application No. PCT/US02/______ (attorney Docket No. 24731-504PC), filed the same day herewith, and reproduced herein).

Initial cell activation was performed using 3:1 ratio between magnetic beads and sorted cells. For re-stimulation, an amount of beads equal to the amount of cells in the culture determined by hand cell count was used. On day 13, 14 or 15 expanded cell cultures were harvested. The cells were counted cells (manual hand count) and the final product was characterized (see, also EXAMPLE 8, which describes growth and preparation of cells using CD3/CD28 antibodies conjugated to colloidal beads (Miltenyi Biotec, Auburn Calif.), and EXAMPLE 9, which describes their use to restimulate resting cells).

F. Phenotyping

For characterization of the final product, the phenotypes of harvested cells were determined, their ability to express IFN-gamma and IL-4 by intra-cellular cytokine staining (ICC) and their production of IFN-gamma, IL-2 and IL-4 (determined by ELISA in the cell culture supernatants of expanded cells before harvesting) were analyzed. Immunophenotyping and ICC experiments were performed as described above. ELISA assays were performed using ELISA kits (R&D, Minneapolis, Minn.) for IFN-gamma, IL-2, IL-4, IL-10, IL-13, TNF-alpha according to manufacturer's recommendations.

G. Preparation of Colloidal Size Microbeads

Paramagnetic colloidal size beads can be purchased from Miltenyi Biotec (Auburn, Calif.; see, also U.S. Pat. No. 6,417,011). As described in U.S. Pat. No. 6,417,011, dextran coated paramagnetic colloidal size particles are prepared by mixing 10 g dextran T40 (Pharmacia, Uppsala Sweden), 1.5 g ferric chloride hexahydrate and 0.64 g ferrous chloride tetrahydrate in 20 ml water and heating to 40° C. The solution is stirred and 20 ml 4 M NaOH is added dropwise with continued stirring. The resulting particle suspension is neutralized with acetic acid, centrifuged for 10 min at 2,000×g, and filtered through a 0.22 μm pore-size filter (Millex GV) to remove aggregates. Unbound dextran is removed by washing in a high gradient magnetic field by washing in columns of steel wool in a high gradient magnetic separation (HGMS) device at a strength of 0.6 Telsa. The particles are washed through the column. These particles can be further derivatized.

### EXAMPLE 2

Restimulation of Rested T-Cells

This example demonstrates that restimulation of previously activated cells that have rested after removal from an activation stimulus results in significant increases in cytokine production compared to the cells that are not re-activated.

Preparation of T-Cells

Pure Th1 cells were prepared by the frequent and repeated activation method as described in EXAMPLE 1 and as exemplified with group 1 cells in EXAMPLE 3 and described in above-noted copending U.S. applications. Briefly, CD4+ cells were purified by positive selection from patients with advanced cancer. The cells were cultured in X VIVO-15 culture medium supplemented with glutamine. On day 10, the cells were incubated with anti-CD3/anti-CD28 conjugated immunomagnetic beads at a 3:1 bead:cell ratio. Every 3 days the cells were restimulated at a 1:1 ratio. On day 14, two days after last stimulation, the cells were harvested and separated from the beads.

Restimulation

The day 14 harvested cells were washed and resuspended in fresh medium. The cells were divided into two groups and each group of cells was incubated for 120 hours. The first group (no restimulation) was cultured without any activation. The second group (restimulation) was restimulated after 72 h (120 h after last stimulation). Samples were taken every 24 h and analyzed by ELISA for cytokine production. For restimulation at 72 h, cells were removed, labeled with anti-CD3 and anti-CD28 mAb and mixed at a 1:3 ratio with freshly collected autologous PBMC.

| IFN-gamma | | | | | |
|---|---|---|---|---|---|
| pg/ml/1 million | 24 h | 48 h | 72 h | 96 h | 120 h |
| No restimulation | 3850 | 2240 | 1050 | 680 | 180 |
| L restimulation | | | 98,800 | 42,500 | 14,600 |
| IL-2 | | | | | |
| pg/ml/1 million | 24 h | 48 h | 72 h | 96 h | 120 h |
| No restimulation | 120 | 60 | <50 | <50 | <50 |
| restimulation | | | 2800 | 580 | <50 |
| IL-4 | | | | | |
| pg/ml/1 million | 24 h | 48 h | 72 h | 96 h | 120 h |
| No restimulation | <26 | <26 | 21 26 | <26 | <26 |
| restimulation | | | 50 | 35 | <26 |

These data demonstrate that restimulation of primed cells that have rested 120 h after removal from an activation stimulus results in significant increases in cytokine production.

### EXAMPLE 3

Activation and Re-Activation in Tumor Microenvironment Inhibits Cytokine Production

This Example shows the effect of re-activation in a tumor environment versus a non-tumor environment, and also shows that the combination of re-activation of cells that have been produced by the multiple activation method produce more cytokines upon re-activation and are show better resistance to a tumor microenvironment. The data also show that T-cells must be re-activated prior to infusion, particularly in order to function productively in an immunosuppressive micro-environment, and explain prior difficulties in adoptive immunotherapy treatment protocols.

CD4+ cells purified from the peripheral blood of a cancer subject were divided in two groups: Group 1 were activated every 3 days for a period of 12 days and harvested on the 15th day. Group 2 were activated only once on day 0 and harvested on the 15th day.

Both groups of cells were then reactivated by with immobilized with anti-CD3/anti-CD28 monoclonal antibodies in culture medium, and incubated in the presence of IL-10 (100 pg/ml), IL-4 (200 pg/ml), IL-6 (100 pg/ml) and TGF-beta (100 pg/ml) to simulate an immunosuppressive tumor microenvironment. As a control, a portion of each group of cells was activated in the absence of immunosuppressive cytokines. The production of IFN-gammã was measured after 24 hours and expressed as production per 10⁶ cells per 24 hours.

| | |
|---|---|
| Group 1 | Group 2 |

| No Tumor | Tumor | No Tumor | Tumor |
|---|---|---|---|
| Cytokines | Cytokines | Cytokines | Cytokines |
| 2400 pg/ml | 2200 pg/ml | 200 pg/ml | 26 pg/ml |

These data indicate that, when reactivated in culture medium, cells that are repeatedly activated (Group 1) produce significantly more interferon-gamma than the same cells that are activated only once (Group 2; 2400 pg/ml by Group 1 cells vs 200 pg/ml by Group 2 cells). When the repeatedly activated cells (Group 1) are immediately transferred to an immunosuppressive cytokine environment (Tumor cytokines) after activation in medium (No Tumor Cytokines), they continue to produce cytokine (2400 pg/ml vs 2200 pg/ml). Cytokine production in the single activated cells (Group 2) is significantly down regulated when first activated in medium (No Tumor Cytokines) and then transferred immediately to an immunosuppressive environment (Tumor Cytokines) (200 pg/ml vs 26 pg/ml).

These data indicate that T-cells for immunotherapy should be repeatedly activated prior to infusion by methods such as is described in U.S. application Ser. Nos. 10/071,016 and 09/957,194, and in International PCT application No. PCT/US02/______ (attorney Docket No. 24731-504PC), filed the same day herewith, and outlined in EXAMPLE 1) as repeatedly activated cells produce more cytokines and are more resistant to the influence of immunosuppressive cytokines. The cells should the be reactivated in the culture medium as described herein prior to infusion.

If the repeatedly activated cells or the single activated cells are re-activated in the presence of immunosuppressive cytokines, however, neither group produces cytokines.

| Group 1 | | Group 2 | |
|---|---|---|---|
| Activation in | Activation in the presence | Activation in | Activation in the presence of |
| medium | of Tumor Cytokines | medium | of tumor cytokines |
| 2600 pg/ml | >2 pg/ml | 90 pg/ml | >2 pg/ml |

This indicates that T-cells activated in the presence of tumor cytokines will not produce cytokines. If they are activated first and then placed in a tumor microenvironment, they will continue to produce cytokines. Accordingly, methods that rely on a mechanism where infused T-cells require activation in-vivo to exhibit an effector function will likely not produce an effect.

These data indicate that T-cells must be re-activated prior to infusion in order to function productively, particularly in an immunosuppressive microenvironment.

### EXAMPLE 4

Summary of Cytokine Production Data of Day 14 Harvested Cells That Were Last Stimulated on Day 9

The cells were removed from the beads on Day 14 and cultured for 24 h. One group was labeled with anti-CD3/anti-CD28 and mixed with autogous PBMC at a 1:2 ratio. A second group was stimulated with anti-CD3/anti-CD28 conjugated beads and a third group was not restimulated. Cytokine production at 4 h and 24 h was analyzed by ELISA.

| | 4 hr | 4 hr | 4 hr | 24 hr | 24 hr | 24 hr |
|---|---|---|---|---|---|---|
| | *IFN-γ | IL-4 | **TNF-α | *IFN-γ | IL-4 | **TNF-α |
| | (pg/ml) | (pg/ml) | (pg/ml) | (pg/ml) | (pg/ml) | (pg/ml) |
| Day 14 | 526.17 ± | 6.3 ± 0.00 | 103.11 ± | 4625 ± | 6.47 ± | 252.10 ± |
| harvested cells | 702.26 | | 35.68 | 877.46 | 0.29 | 77.70 |
| only (HRV) | | | | | | |
| 1:2 | 2502.45 ± | 7.4 ± 2.34 | 408.45 ± | 21982.86 ± | 20.44 ± | 2665.20 ± |
| HRV:PBMC | 3070.93 | | 15.10 | 21013.53 | 18.32 | 432.31 |
| w/sol 3/28 | | | | | | |
| HRV Cell + | 3338.45 ± | 6.79 ± | 517.24 ± | 15920.95 ± | 9/38 ± | 3084.00 ± |
| CD3/CD28 | 3581.14 | 0.98 | 41.22 | 6440.41 | 2.24 | 1756.93 |
| Beads | | | | | | |

*The concentrations of IFN-γ were normalized to 1.0 ± 10⁶ HARVESTED cells/ml.
**The concentrations of TNF-α were normalized to 1.0 ± 10⁶ TOTAL cells/ml.

The sample size (n) was 8 and included 6 normal donors and 2 cancer donors.

These data demonstrate that restimulation of primed, resting cells prior to infusion results in cells with significantly enhanced cytokine production. The amount of cytokine production sufficiently high as to raise concerns about potential toxicity. This experiment demonstrates that for these cells, low amounts of cytokines are produced within the first 4 hours after re-stimulation and cytokine production peaks around 24 hours post re-stimulation.

This indicates that these re-stimulated cells should be infused before 24 hours, and typically before cytokine production starts to increase, which occurred at about 4 hours after re-stimulation of these cells, which have an activated memory phenotype (CD45RO+, CD25+, CD62L^{Lo}). Cells with such phenotype are expected to extravasate and enter areas of inflammation. By administering them by four hours, they will enter the areas prior to peak cytokine production. Local cytokine production is known to be less toxic than systemic cytokine production.

### EXAMPLE 5

CD4+ cells were activated every 3 days with anti-CD3/anti-CD28 conjugated beads. On day 14 the cells were removed from the anti-CD3/anti-CD28 beads, washed and resuspended in either fresh culture medium, infusion medium(saline) or autologous plasma. The cells were cultured for another 24 hours and the amount of cytokine produced over this period determined by ELISA.

The cells were then reactivated by first labeling with anti-CD3 and anti-CD28 mAb and then either mixing with autologous PBMC enriched for the granulocyte fraction collected by leukapheresis or mixing with anti-CD3/anti-CD28 coated beads.

After 24 hours of incubation in saline, viability of the culture had dropped to 42% compared to the viability of 84% of the same cells in medium. No cytokine production was detectable in the saline cultures, while the medium cultures contained 1500 pg/ml of IFN-gamma. Upon re-activation, the cells cultured in saline did not produce detectable amounts of cytokines, indicating that cells for use in adoptive immunotherapy protocols should not be formulated in saline.

The control cultures maintained in medium were reactivated with either anti-CD3/anti-CD28 beads or a 1:3 ratio of cells: autologous PBMC enriched in granulocytes on day 15. Cytokine production was measured at 2 hours, 3 hours, 4 hours and 24 hours.

IFN-gamma (pg/ml)

| | | Anti-CD3/anti- | |
|---|---|---|---|
| Hours | | CD28 | Autologous PBMC |
| 2 | hrs | 5800 | 670 |
| 3 | hrs | 8200 | 700 |
| 4 | hrs | 8250 | 2400 |
| 24 | hrs | 38,850 | 38,355 |

TNF-alpha (pg/ml)

| | | | |
|---|---|---|---|
| 2 | hrs | 0 | 0 |
| 3 | hrs | 580 | 230 |
| 4 | hrs | 540 | 350 |
| 24 | hrs | 5480 | 5760 |

IL-4 was not detectable.

These data demonstrate that anti-CD3/anti-CD28-labeled T-cells are reactivated prior to infusion by mixing with autologous PBMC. The production of cytokines at 24 hr is equivalent to the production stimulated by the same mAbs immobilized on immunomagnetic beads. When the cells were formulated in autologous plasma and re-activated, the cytokine production was maintained.

### EXAMPLE 6

When looking cytokines produced as a function of the ex-vivo immunotherapy process (i.e. from initial culture to harvest to re-infusion) it was found that in culture the cells general increase in cytokine productions. The cells are then harvested from the culture environment, and formulated (i.e., washed and put into infusion medium). Typically there is delay from formulation to infusion, such as for shipping. The problem, as shown herein, is that by the time the patient and cells are ready for infusion, there is no cytokine production. At that point, cell viability is also significantly decreased. This example presents the results of a study to determine how to keep the cells viable and producing cytokines.

Viability Study

Purified CD4+ cells were activated with anti-CD3/anti-CD28 conjugated beads every 3 days for 9 days. On day 12, the cells were harvested, washed and resuspended at 1×10⁸ cells/ml in various infusion media. These formulated cells were stored for 48 hours at either 4° C., 22° C. or 37° C. The cells from each batch were formulated in saline, 5% dextrose, Plasma-Lyte, Normosol or autologous plasma. Samples were taken at 4 h, 12 h, 24 h and 48 h and analyzed for viability and production of interferon-gamma. Each table presents a different formulation of infusion medium, the numbers are the percent viable cells±standard error. The data represent the results of 6 different patients.

| | | | |
|---|---|---|---|
| 4 h | 12 h | 24 h | 48 h |

Saline

| | | | | |
|---|---|---|---|---|
| 37° C. | 72 ± 14 | 58 ± 20 | 42 ± 18 | 22 ± 12 |
| 22° C. | 93 ± 13 | 82 ± 17 | 48 ± 15 | 26 ± 14 |
| 4° C. | 92 ± 6 | 80 ± 12 | 52 ± 18 | 48 ± 20 |

5% Dextrose

| | | | | |
|---|---|---|---|---|
| 37° C. | 68 ± 12 | 62 ± 14 | 50 ± 20 | 35 ± 5 |
| 22° C. | 94 ± 6 | 90 ± 10 | 82 ± 6 | 20 ± 20 |
| 4° C. | 89 ± 9 | 78 ± 20 | 68 ± 18 | 50 ± 12 |

Plasma-Lyte

| | | | | |
|---|---|---|---|---|
| 37° C. | 92 ± 8 | 80 ± 12 | 75 ± 16 | 25 ± 25 |
| 22° C. | 96 ± 4 | 90 ± 8 | 83 ± 10 | 55 ± 18 |
| 4° C. | 94 ± 8 | 92 ± 10 | 84 ±12 | 62 ± 15 |

Autologous Plasma

| | | | | |
|---|---|---|---|---|
| 37° C. | 98 ± 2 | 97 ± 2 | 93 ± 6 | 85 ± 8 |
| 22° C. | 99 ± 1 | 97 ± 2 | 96 ± 3 | 89 ± 6 |
| 4 ° C. | 93 ± 4 | 85 ± 8 | 80 ± 10 | 78 ± 12 |

Normosol

| | | | | |
|---|---|---|---|---|
| 37° C. | 93 ± 7 | 82 ± 14 | 70 ± 12 | 28 ± 16 |
| 22° C. | 93 ± 7 | 85 ± 6 | 78 ± 16 | 58 ± 16 |
| 4° C. | 90 ± 8 | 80 ± 6 | 80 ± 12 | 48 ± 20 |

These results demonstrate that cells formulated in infusion medium exhibit a significant decrease in viability notable within the first 12 h after formulation. Increased temperature results in more rapid loss of viability and decreased temperature slows the loss of viability. Formulation in autologous plasma was capable of maintaining cell viability. In addition, in the cells in group formulated in autologous plasma was the cytokine production maintained. Representative data from one culture is shown below:

IFN-Gamma Cytokine Production at 22° C. (pg/ml)

| | 4 h | 12 h | 24 h | 48 h |
|---|---|---|---|---|
| Saline | ND | ND | ND | ND |
| 5% Dextrose | ND | ND | ND | ND |
| Plasma-Lyle | 240 | 80 | ND | ND |
| Normosol | 280 | 120 | ND | ND |
| Autologous Plasma | 9600 | 6200 | 4800 | 2200 |

*ND = not detectable

As shown in the Example, below, reactivation in infusion medium containing antibody-conjugated colloidal size particles also maintains viabilty, since the particles do not have to be removed prior to infusion.

### EXAMPLE 7

Preparation of anti-CD3 and Anti-CD28 Monoclonal Antibody Colloidal Paramagnetic Beads

Human anti-CD3 and anti-CD28 mouse monoclonal antibodies are immobilized on Miltenyi Goat-Anti-Mouse (GAM) micro-beads for Th1 cell expansion. The beads are used for activation of primed CD4⁺ T cells (CD4⁺ T cells activated using Human anti-CD3 and anti-CD28 immobilized on Dynal beads). Advantages of using these beads include, for example: 1) The Miltenyi beads are micro particles that remain in colloidal suspension, as a result these beads do not settle at the bottom of the flask in bioreactor; 2) Miltenyi micro-particles following binding to CD4 T cells will be internalized or shed, as a result the activation signal through CD3 and CD28 will be transient and not continuous; and 3) the need to debead the product prior to infusion in patients is eliminated.

A. Materials:

Goat anti-Mouse IgG Miltenyi Microbeads

Dulbecco's Phosphate Buffered Saline (dPBS)

General Buffer (dPBS with 1% HSA)

OKT3 human anti-CD3 monoclonal Antibody, 1 mg/ml (Ortho)

CD28 ASR, human anti-CD28 Bulk monoclonal Antibody, 1 mg/ml (BD)

MS or LS column for MiniMACS or OctoMACS (Miltenyi order #130-042-201 or 130-042-401)

MiniMACS (Miltenyi order # 130 042 302)or MidiMACS unit (Miltenyi order #130 042 102)

Sample CD3/CD28 Antibody Solution for Quality Control

Sample CD3/CD28 Expansion Beads for Quality Control

Miltenyi CD3/CD28 T-Cell Expansion Beads

B. Preparation

To prepare the beads, human anti-CD3 and anti-CD28 were mixed at ratio of 1:1 and added to the solution of beads. The mixture of beads and antibodies was incubated room temperature. The beads were washed on a Miltenyi MS column 10 times to remove unbound antibodies and eluted from the column using X-vivo15.

To prepare the volume of beads to be conjugated is selected. Each 2 mL of GAM Miltenyi beads results in about 2 mL of anti-CD3/anti-CD28 beads. Twenty μL of anti-CD3/anti-CD28 beads were required to stimulate ≦10⁷ total cells. The colloidal solution of GAM Miltenyi beads was gently vortexed to re-suspend the beads, which were then transferred to a 12×75 polypropylene tube for coupling the antibodies.

To prepare a CD3/CD28 antibody solution, CD3 and CD28 antibodies were mixed together in equal amounts to produce a homogeneous solution. For each 500 μl of GAM beads 100 μg each of anti-CD3 and anti-CD28 antibody solution was used. The solution was produced by mixing equal amounts of anti-CD3 and anti-CD28 antibodies.

For conjugation of the antibodies to the beads, 200 μl of anti-CD3/anti-CD28 solution was added for every 500 μl of GAM Miltenyi beads and the resulting mixture is gently vortexed. The antibody-bead solution tube was placed on spindle rotors for 60 minutes at room temperature.

To remove the unbound antibody, an MS column was assembled in the magnetic field of an OctoMACS separator (Miltenyi Magnet). A collection tube was placed under the column. 500 μl of degassed PBS buffer was placed on top of the column and run through to pre-equilibrate. The bead-antibody solution was loaded onto the pre-equilibrated column. Antibody-bead solution was run through, and unbound antibody in the effluent was collected. The column was washed with 10×500 μL General Buffer (dPBS with 1% HSA) and total effluent collected as negative fraction (contains unbound antibody). 500 μl of X-Vivo15 was applied to the column and the beads pushed out and stored in a sterile 50 mL conical centrifuge tube at 4° C.

### EXAMPLE 8

Th1 Cell Preparation Using Antibodies Immobilized on Nanobeads for Re-Stimulation

A. Preparation of the Th1 Cells

As in the above Examples, leukocytes (˜5000 ml) were obtained from Donor/Patients by leukapheresis. The leukapheresis product was further purified using magnetic separation techniques, described above, to isolate that CD4 cell fraction (>80% pure). The CD4 cell fraction and anti-CD3/anti-CD28 immobilized Dynal beads were incubated together for 3 days. Briefly, approximately 25×10⁶ purified CD4+cells were placed in a sterile 12×75 culture tube with cap. The cells were centrifuged and the supernatant discarded. The cells were resuspended in 2.5 mL X-VIVO 15 Medium (10×10⁶ cells per mL).

1875 μL of CD3/CD28 coated sheep anti mouse IGg (SAM) Dynabeads (4×10⁷ beads/mL at a 3:1 ratio of beads to cells) were dispensed into a 50 mL conical centrifuge tube, which was placed into the MPC Magnet and rocked gently 5 times to expose all of the liquid to the magnet. At the end of 5 minutes, with the tube on the magnet, the supernatant X-Vivo 15 medium was removed. The tube was then removed from the magnetic field. The beads were gently disturbed by tapping the tube.

The purified CD4+ cells gently mixed with the bead pellet by tapping. The tube was placed on ice for 20 minutes and vortexed gently every 5 minutes during this incubation. 22.5 mL of pre-warmed X-Vivo 15 was added to the 2.5 mL bead/cell mixture for a final concentration of 1×10⁶ cells/mL. These cells were inoculated into a culture bag (LifeCell). The bag was placed in a 37° C. incubator at 5% CO₂ and 100% humidity. The Dynal Beads were removed by magnetic separation resulting in a cell culture mixture.

B. Restimulation and Expansion of Th1 Cells in Bags

The cell culture mixture is re-stimulated with anti-CD3/anti-CD28 immobilized on GAM Miltenyi microbeads (Miltenyi Biotec, Auburn Calif.), prepared as described in EXAMPLE 7. At day 3 (72 hours post initiation, the contents of the bag were gently but thoroughly mixed, and then transferred to a 50 mL conical centrifuged tube, which was placed into the MPC Magnet for 5 minutes. The supernatant was removed and into a fresh 50 mL conical centrifuge tube.

About 5.0 mL of the well-mixed cell suspension was transferred into each of 2 tubes for analyses. The tube containing the bulk of the cell suspension was centrifuged at 1200 rpm for 5 minutes, and the supernatant was transferred into another sterile 50 mL conical centrifuge tube, centrifuged and resuspended in conditioned medium at a cell density of 100×10⁶ cells/mL (WBC count from Sysmex×volume of medium×% viability)/100=mL of conditioned medium to add) and placed on ice.

20 μL of anti-CD3/anti-CD28-GAM-Miltenyi microbeads per 10×10⁶ cells was added, mixed well, incubated on ice for 20 minutes, vortexing gently every 5 minutes during the incubation.

When incubation is complete the density is adjusted by addition of a 25:75 (v/v) mixture of conditioned medium and fresh X-Vivo 15 to 1×10⁶ cells/mL. These cells are inoculated into a new culture bag and incubated. Each day of the incubation, a portion of the working supernate is exchanged for fresh medium to replenish nutrients and remove waste products.

On day 6 and day 9 of the culture, the cell culture mixture is restimulated with anti-CD3/Anti-CD28 immobilized on GAM Miltenyi Micro Beads. 20 μL of anti-CD3/anti-CD28-GAM-Miltenyi microbeads was added per 10×10⁶ cells that had been resuspended at a density of 100×10⁶/mL. The tube containing the cell/bead suspension was placed into an ice bath for twenty minutes and mixed gently every five minutes during the cold incubation, the cells bead mixture is transferred to a culture bag, which was placed in the incubator. On day 13, the cells were harvested.

C. Results

The resulting cells had the following properties, which indicate that they are polyclonal Th1 cells:

A. Purity ˜99% CD4+ and CD3+.

B. Viability >90%

C. Produce a large amount of INF-gamma cytokine (up to about 10 ng per million cells)

D. Do not produce detectible IL-4

E. Do not produce detectible TGF-beta

F. Do not produce detectible IL-10.

G. Do not have detectible CTLA-4 on cell surface.

H. At gene expression level these cells exhibit:

1. detectible expression of INF-gamma, IL-2, IL-15, IL-18, TNF-alpha, TNF-beta.

2. undetectable IL-4, IL-10, IL-5, IL-12P35, IL-12P40, IL-1 beta, IL-150 alpha, IL-6 expression.

### EXAMPLE 9

Reactivation of Cells Using CD3/CD28 Antibodies Conjugated to Colloidal Size Paramagnetic Beads

Resting Th1 cells harvested cells produced by any method can be used. In this example, the Th1 cells were produced by the method in EXAMPLE 7. The cells were washed to remove all supernatant liquid such as medium from the cell culture), and placed in fresh medium and re-stimulated with anti-CD3/anti-CD28 immobilized on Miltenyi micro beads as in EXAMPLE 7.

After the initial incubation, the cells were re-suspended in new clean medium and allowed to incubate 24 hours. The resulting cell culture mixture has all of the characteristics associated with cells when originally harvested. In was found that these cells exhibit augmented INF-gamma production following stimulation (up to 2.5 ng per million cells in 24 hours).

Since modifications will be apparent to those of skill in this art, it is intended that this invention be limited only by the scope of the appended claims.

1. A method, comprising:
re-activating harvested previously activated or primed T-cells, wherein the T-cells are cells for adoptive immunotherapy; and
infusing them into a subject.

2. The method of claim 1, wherein the cells are rested following harvesting.

3. The method of claim 1, wherein the harvested cells are frozen and then thawed prior to reactivation.

4. The method of claim 1, wherein, the cells are activated no more than about 4 hours prior to infusion.

5. The method of claim 1, wherein the cells are rested for about 24 to about 120 hours.

6. The method of claim 1, wherein the cells are rested for about 72 to about 96 hours.

7. The method of claim 1, wherein re-activation is effected by contacting the cells with activating monoclonal antibodies.

8. The method of claim 1, wherein re-activation is effected by contacting the cells with activating monoclonal antibodies; and then
mixing the with peripheral blood monocytes (PBMC).

9. The method of claim 7, wherein the activating monoclonal antibodies are immobilized on cells.

10. The method of claim 7, wherein the activating monoclonal antibodies are immobilized colloidal size particles.

11. The method of claim 10, wherein the colloidal size particles are paramagnetic beads.

12. The method of claim 1, wherein the harvested T-cells are produced by
collecting source material from a subject;
purifying T-cells from the source material; and
activating the T-cells a minimum of 3 times at 2-4 day intervals, whereby a highly pure population of polyclonal Th1 memory cells are produced.

13. The method of claim 1, wherein the T-cells are purified CD4+ cells.

14. The method of claim 13, wherein the CD4+ cells are purified by positive selection

15. The method of claim 14, wherein the CD4+ cells are purged of CD45RO+ cells

16. The method of claim 12, wherein the source material is purged of platelets

17. The method of claim 15, wherein the source material is purged of platelets

18. The method of claim 12, wherein the source material is purged of monocytes.

19. The method of claim 17, wherein the source material is purged of monocytes.

20. The method of claim 12, wherein the initial activation of the T-cells is effected by contacting the cells with immobilized anti-CD3 and anti-CD28 mAbs.

21. The method of claim 20, wherein the anti-CD3 and anti-CD28 mAbs are immobilized on immunomagnetic beads.

22. The method of claim 20, wherein the anti-CD3 and anti-CD28 mAbs are immobilized on colloidal size particles.

23. The method of claim 1, wherein:
the T-cells are rested for 72-120 hours after harvest;
labeled with monoclonal antibodies; and
mixed with autologous peripheral blood monocytes (PBMC) prior to infusion.

24. The method of claim 1, wherein:
the T-cells are rested for 72-120 hours after harvest;
contacted with immobilized activating antibodies; and
formulated for infusion.

25. The method of claim 24, wherein the cells are forumlated in infusion medium.

26. A composition of T-cells, comprising T-cells suspended in plasma, wherein the plasma is autologous with respect to the T-cells.

27. The composition of claim 26, wherein the cells are suspended at a density of at least about 10⁷ cells per ml.

28. The composition of claim 26, wherein the cells are suspended at a density of at least about 10⁸ cells per ml.

29. The composition of claim 26, wherein the T-cells are labeled with monoclonal antibodies.

30. A composition of T-cells, comprising T-cells; and
anti-CD3 and anti-CD28 mAb-conjugated colloidal size particles.

31. The composition of claim 30, wherein the cells at a density of at least about 10⁷ cells per mi.

32. The composition of claim 30, wherein the cells at a density of at least about 10⁸ cells per ml.

33. The composition of claim 30, wherein particles are dextran coated.

34. A method for extending the shelf-life of T-cells for adoptive immunotherapy, comprising suspending the T-cells in autologous plasma.

35. The method of claim 1, wherein the harvested cells comprise at least 50% Th1 cells.

36. The method of claim 1, wherein the harvested cells comprise at least 70% Th1 cells.

37. The method of claim 1, wherein the harvested cells at least 50% Th2 cells.

38. The method of claim 1, wherein the harvested cells at least 70% Th2 cells.

39. The method of claim 8, wherein the PBMC are autologous with respect to the T-cells.

40. The method of claim 8, wherein the PBMC are allogeneic with respect to the T-cells.

41. A composition produced by the method of claim 7.

42. A composition produced by the method of claim 35.

43. A composition, comprising activated T-cells and autologous peripheral blood monocytes (PBMC).

44. The composition of claim 43, wherein the PBMC are autologous with respect to the T-cells.

45. The composition of claim 43, wherein the PBMC are allogeneic with respect to the T-cells.

46. The method of claim 1, wherein the cells are infused into a patient from whom the peripheral blood monocytes (PBMC) were removed.

47. The method of claim 5, wherein the cells are infused into a patient from whom the peripheral blood monocytes (PBMC) were removed.
